(19) United States
(12) Patent Application Publication
(10) Pub. No.: US 2016/0370380 A1
(43) Pub. Date: Dec. 22, 2016

(54) COMBINATORIAL ANTIBODY DIAGNOSTIC

(71) Applicant: PharmaSeq, Inc., Monmouth Junction, NJ (US)

(72) Inventors: Wlodek Mandecki, Princeton, NJ (US); Ji Li, Hightstown, NJ (US); Maryann Gruda, Yardley, PA (US)

(21) Appl. No.: 15/184,630

(22) Filed: Jun. 16, 2016

Related U.S. Application Data
(60) Provisional application No. 62/180,362, filed on Jun. 16, 2015.

(51) Int. Cl. G01N 33/68; G06F 19/16; G06F 19/18; G01N 33/574; G01N 33/564

(52) U.S. Cl. CPC G01N 33/6845; G01N 33/6854; G01N 33/574; G01N 33/564; G06F 19/18; G06F 19/16; G01N 2800/24; G01N 2800/26

(57) ABSTRACT

Provided among other things is an indexed library on one or more solid phase supports of a substantial representation of all theoretical peptide combinations having a certain length of 3 to 5 amino acids, or a combination thereof, and being formed with a certain collection of amino acids that numbers as follows:

| | # of amino acids |
|---|---|
| Length | in collection |
| 3 | 6 to 18 |
| 4 | 4 to 18 |
| 5 | 4 to 18 |

the peptides spaced apart from the supports sufficiently such that one or more of the peptides binds an antibody composition substantially more strongly than others

This application claims the priority of U.S. Ser. No. 62/180,362, filed Jun. 16, 2015, the contents of which, including its appendices, are incorporated herein in their entirety.

The present application relates generally to short, diverse peptide libraries, and methods of characterizing biomolecules such as antibody compositions.

Heretofore methods of characterizing antibody or other antibody binding patterns have used arrays of relatively large peptides, typically made with a close to complete repertoire of naturally occurring amino acids. Now provided is a method of characterizing biomolecules with very short peptides, which can be built from a smaller repertoire of amino acids. Moreover; with such a repertoire, the array can provide a substantial representation of all possible peptide sequences.

Even with the apparently limited size of the peptide repertoire, it can be used to characterize whether a subject (e.g., animal) has a disorder or indication. The repertoire can further be used to characterize biomolecules.

Further provided herein is a computer-implemented method of analyzing biomolecules.

### SUMMARY

This invention described herein is of methods of analyzing biomolecules using peptide libraries and methods of forming such libraries. Included is an indexed library comprising features from the independent claims, and methods for using such libraries, substantially as shown in and/or described in connection with at least one of the figures, are disclosed. Various advantages, aspects, and features of the present disclosure, as well as details of illustrated embodiments thereof, will be more fully understood from the following description and drawings. The foregoing summary is not intended, and should not be contemplated, to describe each embodiment or every implementation of the present invention. The Detailed Description and exemplary embodiments therein more particularly exemplify the present invention.

### DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 schematically shows two solid phase particles 10, each with a linker Lk, a spacer Sp, and a 4-residue peptide;

FIG. 2 shows sequence alignments from Example 2; and

FIG. 3 shows competitive binding from Example 3.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

An indexed library of peptides is one where the identity of a member peptide can be determined by methods other than directly determining the sequence of the peptide. The method of identification can be for example by location of the binding on a solid support (solid phase support), or by separate solid supports (particles) corresponding to separate peptides having identifiable characteristics. Indexing includes separate sold supports that are further indexed by location. Those identifiable characteristics can for example be a visual pattern, or coordinates of a location within a microarray, or an electromagnetic signature. In embodiments, several to thousands of peptides are on one solid phase support and are indexed by location. In embodiments, a given solid phase particle has substantially one peptide (substantially meaning that there can be a small amount, normal to the chemistry, of erroneous syntheses).

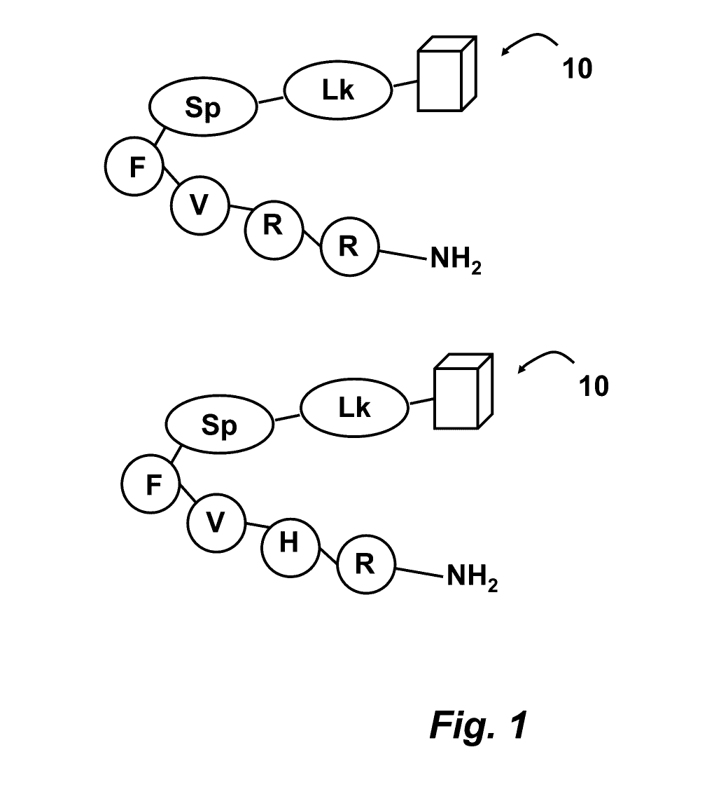

FIG. 1 schematically shows two solid phase particles 10, each with a linker Lk, a spacer Sp, and a 4-residue peptide.

In embodiments, the indexed library of peptides includes a substantial representation of all peptide combinations having a certain length of 3 or more amino acids and being formed from a collection of selected amino acids that number 4 or more (“AA diversity”). For example, in embodiments the length can be 3 to 7 amino acids, such as 4 to 7 or 4 to 5 amino acids. Where the amino acid diversity is lower, peptides can be longer, such as 12-mers. The relationship between length and the limit on amino acid diversity is set forth in the table below. By a substantial representation of all combinations it is meant that the library includes more than 50% of the combinations.

Prior to the current invention it is believed to be unknown that a collection of short peptides would provide adequate binding affinities to investigate an antibody composition.

Prior to the current invention it is believed to be unknown that a collection of short peptides based on the following:

| Length | AA Diversity |
|---|---|
| 3 | 6 to 18 or 20 |
| 4 | 4 to 18 or 20 |
| 5 | 4 to 18 or 20 |

would provide a diagnostically significant pattern of binding affinities with respect to an antibody or other biomolecule composition. In embodiments, AA diversity is from 4 to 12 (or 6 to 12 for length=3). In embodiments, AA diversity is 6 to 12, or 6 to 10.

In embodiments, the library of four residue long peptides (where the amino acids are selected from a group of eight amino acids) has a theoretical full diversity of 4,096 peptides, and an actual diversity of about 3,000 or more peptides (that diversity that actually results from constructing the library). In embodiments, a similar library but having peptides that are 5 amino acids long, has a theoretical full diversity of 32,768 peptides, and an actual diversity of 24,000 or more peptides.

In embodiments, the library has a high diversity up to or close to theoretical (e.g.) 4,096 peptides. In embodiments, the library has a high diversity up to or close to theoretical 32,768 (such as 32,000 or more).

A given peptide binds somewhat or substantially more strongly than other peptides in the library when it produces a binding signal that is two times or more higher (after deduction of background). In embodiments, the binding signal is 3, 4 or 5× higher.

In embodiments, the peptides of the library are short. For the purposes of this application, the length does not include the length of relatively non-distinct peptides or amino acids used as part of a spacer. Where the spacer peptides or amino acids are either constant among the members of the library, or are relatively indistinct across the library, one of skill will recognize that other, diverse peptide segments are configured to provide distinct binding to antibodies.

In embodiments, the peptides are synthesized on a solid phase (e.g., solid particles or microarray). In embodiments, the synthesis in place is recognizable by clean C-terminal attachment to the particles, even with peptides that have functionalities that would preclude clean attachment to the C-terminal post peptide synthesis).

In embodiments, the collection of amino acids contains two or three of the aromatic standard amino acids (2 or 3 of F, W or Y). (The standard amino acids are G, A, V, L, I, P, M, K, R, H, Y, F, W, D, E, N, Q, T, S and C.) In embodiments, the collection of amino acids contains all three of the aromatic standard amino acids.

In embodiments, the collection of amino acids contains two or three of the positively charged standard amino acids (positively charged with respect to the side chain at neutral pH) (of K, R or H). In embodiments, the collection of amino acids contains all three of the positively charged standard amino acids.

In embodiments, the collection of amino acids contains two or more of the standard amino acids with aliphatic side chains of 3 or more carbons (of V, I, L). In embodiments, the collection of amino acids contains V and I or L. In embodiments, the collection of amino acids contains V and I.

In embodiments, the amino acids are natural amino acids or substantially natural amino acids. Natural amino acids are those 20 encoded by the codon usage code. A collection of amino acids is of substantially natural amino acids if 75% or more are natural amino acids, and the others differ only by conservative substitutions (such as up to two additions or deletions of methyl or methylene to non-aromatic side; substitutions to aromatic groups that do not take up substantially more space than two nitro substitutions; shifting of the ring location of hydroxy in tyrosine; substitution of imidazole ring with a 5-6 ring member aromatic ring having one or more nitrogen ring atoms; substitution of 6-member aromatic ring with 5 to 6 member non-basic aromatic; substitution of indole with a 9-10 ring atom planar ring system with an aromatic component; substitution of guanidine with imidamide)

In the simplest embodiments of the invention, where the library is composed or three amino acid residue long peptides, the complete data set for the results encompasses 4 dimensions: a coordinate for the amino acid residue type at each of 3 sequence locations, and a coordinate for the fluorescence intensity, which is related to the affinity of the peptide to the antibody.

The biomolecules examined with the diagnostic methods described herein can be antibodies (monoclonal or polyclonal), receptors, enzymes, nucleic acids, and the like.

Most commonly, binding is scored by measuring a fluorescence intensity associated with a given element of the indexed library. Typically, the binding of biomolecule A to a peptide is associated with fluorescence by further binding a fluorescently labeled detecting biomolecule that has affinity, such as high affinity, for the class of biomolecules under analysis. For example, for a class of antibodies, antibodies to a portion of the antibody away from the active pocket(s) can be used (such as against the Fc region). Similar approaches can be taken with other classes of biomolecules. Or, binding moieties can be chemically coupled to the biomolecule. For example, biotin or avidin can be coupled, and the cognate binding partner, with fluorescent label, later used to confirm binding.

In one embodiment, the solid phase particles used for the library are light-triggered microtransponders (“MTPs”). For example, the MTPs can be triggered by and powered by light, and emit a radio signal that provides an identifying number. Such MTPs are described for example in U.S. Pat. No. 7,098,394 (the description of which MTP is incorporated herein in its entirety). Or, for example, the MTPs can be triggered by and powered by light, and emit a light signal that provides an identifying number (Light-in, light-out MTPs, or “all-optical” MTPs). Such all-optical MTPs are described for example in U.S. patent application Ser. No. 14/631,321 (the description of which all-optical MTP is incorporated herein in its entirety). MTPs can be made to the size of approximately 0.25 mm×0.25 mm by 0.1 mm, or smaller. All-optical MTPs can be made even smaller. As such, they can be used in very small volumes of amino-acid addition reagents. Light-in, Rf-out MTPs are sold by PharmaSeq, Inc., 11 Deer Park Dr., Monmouth Jct., N.J., as p-Chip transponders.

Methods for providing attachment chemistries on the surfaces of MTPs are described for example in U.S. Pat. No. 8,785,352 (the description of which chemistry is incorporated by reference in its entirety).

A very useful tool for making the indexed libraries of the invention on MTPs (including all-optical MTPs) is a Spin Reader described in U.S. Pat. Publ. 2014/0106470 (the description of which spin reader and an assay reader are incorporated by reference in its entirety). In a peptide synthesis, a pool of particles can be reacted with a reagent to add one amino acid, and then the pool is split, and other amino acids are added to the separate pools. With the Spin Reader, the identities of all the particles designated for a given reaction can be read and recorded. When a pool is split, the identifiers for particles getting a given amino acid can read and recorded. Reading can be done before, during or after adding the next amino acid. (The Spin Reader could be used to host the reaction, though in embodiments its volume may be too high for optimal use of reagents.) By serially making appropriate splits (with separate splits joined when to be reacted with the same reagent), one can conduct a protocol that in theory makes most of the theoretical combinations.

The principle of the Spin Reader is that in an appropriately modulated spinning compartment to which excitation signal is sent from an exterior laser, the light-responsive face of a given particle will generally align with the laser so that the MTP's signal is emitted within a relatively short period of time. To the extend that any particles are not read, the lack of a read for that synthesis cycle is recorded in the database, such that this MTP is flagged ill-defined for peptide sequence—and binding results for it are generally disregarded.

The simple principle of the Spin Reader allows ready scale-up. For example, multiple Spin Readers can be used, or multiple triggering lasers can be used in a single Spin Reader. For the all-optical system, the triggering lasers may be more closely spaced, as output signal will be less susceptible to overlap.

Alternatively, the synthesis method outlined in U.S. patent application Ser. No. 14/972,659, filed Dec. 17, 2015 (genomic-scaled nucleic acid synthesis, and other combinatorial syntheses), can be used. In that method, multiple binary separators are used to specifically direct pre-identified particles into the collection destined for a particular synthesis step. After say the addition of the third amino acid, the particles can be pooled, and again pre-identified particles can be directed to the intended reaction pool. The contents of this application are incorporated herein in their entirety.

With MTPs, the binding analysis can be conducted with the assay reader described in described in U.S. Pat. Publ. 2014/0106470.

An exemplary synthesis route starts with the particles as modified to provide reactive amines, such as by the method of U.S. Pat. No. 8,785,352 (incorporated herein in its entirety). An FMOC protection scheme for the terminal amines can, for example, be used. Since these cleave in base, acid labile (such as TFA labile) side chain protecting groups can for example be used. In embodiments, a cleavable linker is attached to the particles. This allows the peptide to be removed for analysis, as desired. The cleavable linker can be selected to withstand the base-induced cleavage of FMOC amino protecting groups, and the deprotection conditions for the side change protection.

As outlined above, typically a spacer group is included that provides little specificity for peptide recognition. For example, to glycines (no side chain) can be used as the spacer.

The new method of identifying diseases with an immune response is but one method of many for identifying such disease. These can be tested for by prior art methods by for example testing for representative proteins and protein segments.

Immunosignature of Antibody in Fitness Space.

The results of an assay involving a peptide library and a target antibody (or biomolecule) can be represented as a dataset in an antibody “fitness space” defined as a multi-dimensional sequence space over which a fitness function (binding affinity) is defined. For a useful peptide library on MTPs, the fitness space is 5-dimensional, with four dimensions corresponding to the amino acid in the first, second, third and fourth position in the peptide sequence, and the fifth dimension being the fluorescence intensity (corresponding to the antibody binding propensity to that peptide). To establish relationships between the points in the fitness space, a distance function is commonly introduced. The Hamming distance is the total number of as sequence differences between two aligned sequences. If two aligned peptide sequences differ in N positions, the Hamming distance is N. For instance, the Hamming distance between YHYY and YHYW is 1. An advantage of such a fitness space is the ability to analyze its topological (geometrical) properties, such as local maxima and minima, ridges, valleys, etc. At the local maximum, for all neighbors of the given sequence that are distant by 1, the signal is less than the signal for the given sequence. From a structural perspective, the local maximum represents a sequence which is optimized (in its immediate neighborhood) to bind to the antibody binding site, effectively becoming a “mini-consensus” sequence.

In the above fitness space; a “ridge” point can be defined as a point at which, for a certain position in the peptide sequence, all substitution with a single different amino acid result in a reduced fitness, that is, a lower affinity or fluorescence intensity. Similarly, at a “valley” point, all substitution with a single different amino acid result in an increased fitness.

A set of descriptors associated with of points in the fitness space, the descriptors being the local maximum, the local minimum, the ridge point, the valley point and associated or derivative descriptors, are key characteristics of the antibody binding pattern.

Disease State Diagnosis.

Disease states can be characterized by having a different distribution of peptide binding moieties in blood or other bodily fluids. For example circulating auto-antibodies can be indicative of a tumor (one mix of peptide binding affinities) or of an auto-immune disease (another mix of peptide binding affinities, or the like.

The peptide binding approach with short peptides can highlight the presence of disease-indicative binding moieties, separating these from the complex animal proteome.

In one embodiment, the method is used to identify breast cancer immunoprofiles. These may overlap, but can have specific differences between specific types of breast cancer.

In one embodiment, the method is used to identify pancreatic cancer immunoprofiles. These may overlap, but can have specific differences between specific types of pancreatic cancer.

Identifying Binding Site Peptide Sequence Motifs.

Binding sites on proteins tend to form at specific one or two or three stretches of amino acid sequence (or more, but still a bounded universe). For example, in antibodies, binding is determined by six loop regions, three each on the heavy and light chain.

Binding to a short peptide library to a protein is computationally analyzed. For example, one uses CABS-dock software to analyze the bindings of the array of peptides versus an array of protein binding site sequences. (CABS-dock software: See 32. Kurcinski et al. (2015) CABS-dock web server for the flexible docking of peptides to proteins without prior knowledge of the binding site. Nucleic Acids Res. 2015 May 5.)

Then, the actual binding of the protein to the short peptide library can be experimentally analyzed as described herein. The actual binding patterns are compared to the theoretically calculated ones, to identify one or a few candidate binding structures.

The method can include for example Step 1: Define the structures of possible antibody binding sites. At least two approaches can be applied here:

1a. Generate a large number of amino acid sequences comprising the binding site of the Fv domain of the antibody and then create structural models of the binding site using available software, such as described in Marcatili et al. Antibody structural modeling with prediction of immunoglobulin structure (PIGS). Nat Protoc. 2014 December; 9(12):2771-83.

1b. Create possible binding sites by placing functional groups found in the amino acids and peptides on a grid. The total size of the grid would correspond to the dimensions of a typical binding site on an antibody, which is on the order of 15 Å×15 Å×7 Å.

For either Step 1 a or Step 1 b, the sufficient number of structures can vary from 10³ to 10⁶.

For binding sites with known sequence frameworks, and known programs for converting sequence to structure of the binding site, still a repertoire of binding sites can be generated by accepting multiple structures within a range of probabilities. (As such, the method can be used to provide a chemical measure of probability on top of the computer-generated measure.)

Step 2 can be for example docking each of e.g. 4,096 peptides to each of the binding sites created in Step 1. For example, the peptide-antibody docking software, CABS-dock, is available on the internet (biocomp.chem.uw.edu.pl/CABSdock/ (accessed on Jun. 7, 2015)). The software allows one to determine the energy of the interaction [Blaszczyk et al, Modeling of protein-peptide interactions using the CABS-dock web server for binding site search and flexible docking. arxiv.org/abs/1505.01138 (accessed on Jun. 7, 2015)] and, therefore, to estimate the expected signal strength that would be obtained from the immunoassay results. The output of the computation is the theoretically predicted immunosignature. The output contains the computed immunosignature for each of the antibody binding sites from Step 1.

Step 3 can be for example comparing the experimentally determined immunosignature to the set of computationally determined immunosignatures. The best-fitting computed immunosignature is identified by calculating the RMS deviation or the correlation coefficient between the experimentally determined immunosignature and each of the computed immunosignatures in the set. This best-fitting computed immunosignature directly identifies the 3D structure (which was used to obtain the particular computed immunosignature), and this 3D structure becomes the predicted structure of the antigen-binding site on the antibody. It is possible that more than one adequate structural fit is obtained.

The method allows to obtain the structure of the antigen binding site without x-ray crystallography or NMR analysis.

The method allows one to associate the 3D structure of the binding site on an antibody with its immunosignature. This has profound implications. This 3D structure, in turn, can identify the antigen that the given antibody recognizes, as there is a clear structural complementarity between the binding site on the antibody and the structure of the antigen. 3D structures of many antigens are known (see entries in the Protein Data Bank). Knowing the antigen may identify a disease (type of infectious agent, type of cancer, type of allergy, neurogenerative disorder, others) based only on the immunosignature.

Other proteins, for instance, a protein receptor of therapeutic significance could be subjected to this immunosignature analysis. Applying the above described method yields the structure of the binding site on the receptor. This, in turn, can be used for screening chemical compounds to obtain drug leads.

Utilizing short binding peptides (such as 3 to 5 or 2 to 4) can reduce the conformational complexity of the peptides in the computer-generated docking testing. Short binding peptides can otherwise also reduce data complexity.

Specific embodiments according to the methods of the present invention will now be described in the following examples. The examples are illustrative only, and are not intended to limit the remainder of the disclosure in any way.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more.

Where a sentence states that its subject is found in embodiments, or in certain embodiments, or in the like, it is applicable to any embodiment in which the subject matter can be logically applied.

### Example 1

### Synthesis and Characterization of a Randomized Peptide Library

A total number of ˜20,000 MTPs were used in this work. The chips were separated in 8 vials, each with 2,500 MTPs. IDs of chips in each vial were read on the Spin Reader, and recorded in a database file. Vials #1-8 were used for synthesis of random peptide library and the ninth vial was used for synthesis of an Influenza Hemagglutinin (HA) peptide sequence for QC purpose. The known sequence is NH2-YPYDVPDYA-Spacer-cleavable_linker-pChip (9 aa). The spacer has sequence GG.

Before creating the random peptide library, a spacer sequence GG was first synthesized on polymer coated MTPs. Then a random 4-residue peptide library using 8 selected amino acids (Arg (R), His (H), Ile (I), Lys (K), Phe (F), Tip (W), Tyr (Y), Val (V)) was synthesized by split-and-pool strategy. In this work 4 amino acids were used, specifically the following eight “grand catcher” amino acids reported by Bachi et al. (Anal Chem 80(10):3557-3565, 2008) in the random sequence: The eight “grand catchers” were found to be able to bind significantly more cytoplasmic proteins in red blood cell (RBC) lysate than the other amino acids (Bachi et al.).

The split-and-pool synthesis typically consists of three steps: splitting, separate coupling of a single amino acid per reaction, and recombining the samples to randomize the particles to prepare for the next round. IDs of MTPs were recorded using the PharmaSeq spin reader at each split step so that the synthesis history can be tracked and recorded for each chip. The split-and-pool synthesis was performed for 4 rounds so that the final peptides consist of 4 amino acids. The overall theoretical size of the final peptide library is 4,096 (ie., 8⁴) with about 5 replicates for each peptide, i.e. a total population of about 20,000 chips.

All MTP IDs were read by a Spin Reader that allows MTPs to be quickly read and recorded by PharmaSeq ID readers (wands) when they are spinning in solvents in an Eppendorf tube. In general, there is about 0.7%-0.9% loss/damage rate after each round of synthesis. The same loss/damage rate was also observed for vials #1-8, The reason could be either loss or damage of chips, or simply because, if the read time is limited, the spin reading step is unable to cover 100% of all chips, though it recovers most.

To confirm the chemistry, a 9-aa peptide from Influenza Hemagglutinin (HA) was synthesized under the same conditions. The quality of the control peptide was checked by mass spectroscopy following cleavage from the MTPs. MS data confirmed that the peptides synthesized on MTPs had the correct molecular weight and the synthesis is relatively clean (data not shown). The control MTPs were tested in HA peptide assay, and found to be specifically recognized by anti-HA antibody, while random peptides or no peptides (polymer only) groups had very minimum affinity to anti-HA antibody (data not shown). The results of MS and HA peptide assay in the #9 control group confirms that the procedure we were using successfully added the desired peptide onto MTPs, thus the successful chemistry (conducted concurrently) implies the success of the syntheses in the other vials (vials #1-8).

In vial #1, the average percentage of MTPs present in successive synthesis rounds was 12.25%, almost the theoretical value of 12.5%. Thus, the splitting process was highly random.

### Example 2

### Antibody Binding

The MTP peptide library was tested in a relatively less complex system, i.e., in buffer system spiked with a known antibody. The anti-HA antibody (Sigma) used was generated against an 9-aa peptide (YPYDVPDYA).

MTPs with synthesized peptides were pre-incubated with Membrane Blocking buffer (Invitrogen) for 30 min twice at RT and washed by Tris-buffered saline with 0.05% Tween-20 (TBST) three times. Then the MTPs were incubated with 10 □g/mL biotinylated anti-Hemagglutinin (HA) monoclonal antibody (Sigma-Aldrich) in LowCross Buffer (Candor Bioscience) for 1 hour at RT. The MTPs were subsequently washed with TBST three times and incubated with 10 ug/mL streptavidin-phycoerythrin (SAPE) conjugate in LowCross Buffer for 1 hour at RT in the dark. Following incubation, the MTPs were washed with TBST twice and analyzed by PharmaSeq flow reader at the wavelength of 532 nm. The average fluorescence intensity of labeled antibody binding to the MTPs is quantitatively determined and associated with their IDs as the chips pass in planar suspension through a flow cell (Simuplex™ flow reader from PharmaSeq). Each MTP was read ≧10 times during multiple passes through the flow reader to reduce the coefficient of variation (CV).

The assay signals of MTPs were analyzed and associated with the chip IDs using the FlowWorkshop software (PharmaSeq). Then a database of the 4-residue peptide sequences with associated MTPs IDs was built using an add-on feature of FlowWorkshop. The principle of this feature is to track the ID of each MTP at each synthesis step in each synthesis vial during the split-and-pool process so that it is able to provide the synthesis history for each D. The two databases (assay signals with Ds and peptide sequences with IDs) were then integrated to provide the information of peptide sequence and its associated assay signal.

Results, A total of 5,307 random 4-aa peptides were tested in the assay and we found that different peptides behaved quite differently when binding to anti-HA antibody (data not shown). The majority of tested MTPs showed low assay signal, thus low binding to anti-HA antibody. A few MTPs exhibited high binding to anti-HA antibody in the assay.

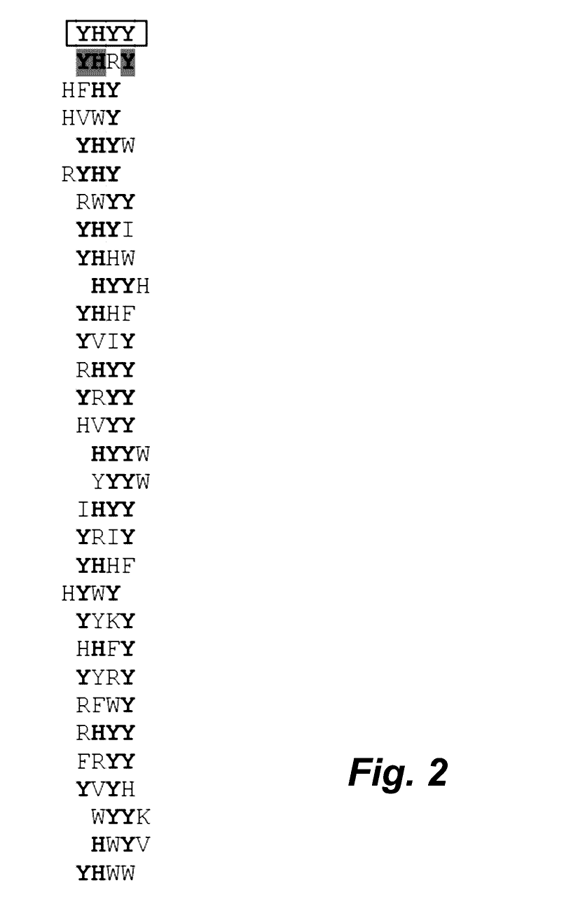

The peptide sequence on each MTP was retrieved by PharmaSeq software FlowWorkshop so that a binding signature to anti-HA antibody could be analyzed. Statistics showed that the occurrences of each amino acid used in the synthesis are not equally distributed when chips with top 30 scoring sequences (with respect to fluorescence intensity) were analyzed (FIG. 2, sequences aligned). Amino acid Y and H have significant higher occurrences than the rest. We extended the analysis to all 5,307 chips to see if there is any binding preferences of anti-HA antibody. When the chips were sorted based on the assay signal on them, we found that the occurrences of each amino acid are highly biased in chips with strong signal and weak signal. Again, amino acid Y and H showed very high occurrences in chips with strong signal while Y has very low occurrence in chips with low signal. In contrast, amino acid K frequently shows up in chips with low signal.

### Example 3

### HA Competitive Binding by Deduced Signature Sequence

With additional studies similar to the above using dimer and trimer libraries, a signature binding sequence of YHYY was deduced.

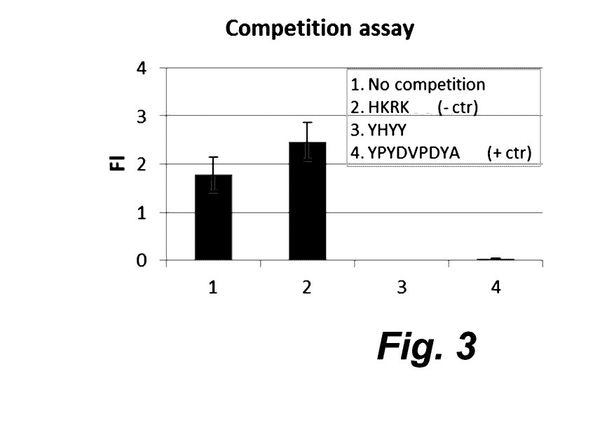

A validation assay used the signature peptide to compete with the original epitope (YPYDVPDYA) that was used to generate the antibody. The results in FIG. 3 show that large amount of the free YPYDVPDYA-Spacer peptide in the solution competed with the same peptide on the MTPs that resulted in reduced assay signal. The hypothesized 4-aa signature sequence (YHYY) with spacer GG also competed well (FIG. 3). As a negative control, 4-aa peptide HKRK with spacer GG showed no competitive activity, HKRK is selected as a negative control because it was found to have very low binding to anti-HA antibody in the screen assays with more than 5,000 MTPs discussed above. Taken together, the results suggested that YHYY has high binding affinity to the anti-HA antibody and thus it is validated to be the 4-aa binding signature sequence of the anti-HA antibody in the library.

### Example 4

### Biomolecule Stripping, Reuse

Unlike peptide microarray or any other techniques that involve peptide printing in which the peptides are passively absorbed on the solid surface, the peptides on MTPs are chemically synthesized with covalent bonds bound to the chips. Theoretically the peptides on MTPs are very stable and may be used multiple times without compromising the performance of the assay.

Procedures.

MTPs from the anti-HA binding assay were subjected to a striping process to remove biomolecules bound onto the peptides on the MTPs. The purpose of striping was to recycle the MTPs in another assay. To strip the bound antibody and SAPE from peptides on MTPs, the MTPs were first incubated with 3.5M MgCl2 for 30 min and then 0.1M citric acid for 30 min at RT. The treated MTPs were washed by TBST three times and analyzed by PharmaSeq flow reader to verify the stripping results. The stripped MTPs were then used in a repeated anti-HA binding assay.

The assayed MTPs were stripped and tested again in the assay. After striping, the binding antibody and SAPE were totally removed from the binding peptide (data not shown) so that the MTPs could be reused in the second assay. In most cases MTP showed the strong signal in the 1st assay also had strong signal in the 2nd assay. The correlation coefficient R² is 0.7894 between two assays in the x-y log-log plot. A noticeable group of MTPs lost signal in the 2nd assay. Signal loss might due to the loss or damage of polymer or peptides during stripping step. However such a group is relatively small (about 100 chips compared to 5,307 chips in total) and thus, it didn't change the main conclusion.

This invention described herein is of a method of characterizing binding moieties, a method of identifying disease states, and a method of determining binding site structures. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

Additional embodiments include:

### Embodiment 1

An indexed library on one or more solid phase supports of a substantial representation of all theoretical peptide combinations having a certain length of 3 to 5 amino acids, or a combination thereof, and being formed with a certain collection of amino acids that numbers as follows:

| | # of amino acids |
|---|---|
| Length | in collection |
| 3 | 6 to 18 |
| 4 | 4 to 18 |
| 5 | 4 to 18 |

the peptides spaced apart from the supports sufficiently such that one or more of the peptides binds an antibody composition substantially more strongly than others.

### Embodiment 2

The indexed library of Embodiment 1, wherein peptides of the library are made with substantially natural amino acids.

### Embodiment 3

The indexed library of Embodiments 1 or 2, wherein peptides of the library are on separate solid phase particles.

### Embodiment 4

The indexed library of Embodiment 3, wherein peptides of the library are indexed by having their respective solid phase particles be part of or connected to light-responsive transponders.

### Embodiment 5

The indexed library of Embodiments 1 to 4, wherein peptides of the library are made with 8 to 10 amino acids, including Arg, His, Ile, Lys, Phe, Trp, Tyr and Va.

### Embodiment 6

A method of characterizing a first antibody composition comprising contacting the antibody composition with the indexed library of Embodiments 1 to 5, measuring the binding of the antibody composition to a substantial representation of the theoretical peptides combinations of the indexed library, and calculating a measure of similarity to such measurements with one or more comparative antibody compositions

### Embodiment 7

The method of characterizing of Embodiment 6, wherein the measurement comprises identifying Hamming distance=1 local maximums.

### Embodiment 8

The method of characterizing of Embodiment 6, wherein the method distinguishes different monoclonal antibodies that bind the same or an overlapping site on an antigen.

### Embodiment 9

The method of identifying an antibody by comparing the immunosignature to a database of immunosignatures.

### Embodiment 10

A method of identifying a high probability for the existence of a disease with an immune response in an animal comprising: (A) contacting antibodies collected from a bodily fluid of the animal with an indexed library of a substantial representation all theoretical peptide combinations having a certain length of 3 to 5 amino acids, or a combination thereof, and being formed with a certain collection of amino acids that numbers as follows:

| | # of amino acids |
|---|---|
| Length | in collection |
| 3 | 6 to 18 |
| 4 | 4 to 18 |
| 5 | 4 to 18 |

(B) measuring the binding of the antibody composition to substantially all of the peptides of the indexed library to derive a binding pattern, and (C) comparing the binding pattern with reference binding patterns from animals known to have the disease and determining if there is sufficient similarity to one or more reference binding patterns to identify said high probability.

### Embodiment 11

The method of Embodiment 10, wherein the disease is a cancer.

### Embodiment 12

The method of Embodiment 10, wherein the disease is an autoimmune disease.

### Embodiment 13

The method of Embodiment 10, wherein the disease is a microbial infection. (For the purposes of this application, a microbial infection is a viral, bacterial or fungal infection.)

### Embodiment 14

The method of Embodiment 10, wherein the disease is an allergy.

### Embodiment 15

A method of testing the quality of a test biomolecule (binding moiety) composition comprising: (i) contacting the test binding moiety composition with an indexed library of a substantial representation of all theoretical peptide combinations having a certain length of 3 to 5 amino acids, or a combination thereof, and being formed with a certain collection of amino acids that numbers as follows:

| | # of amino acids |
|---|---|
| Length | in collection |
| 3 | 6 to 18 |
| 4 | 4 to 18 |
| 5 | 4 to 18 |

(ii) measuring the binding of the test biomolecule composition to substantially all of the peptides of the indexed library to derive a binding pattern, and (iii) comparing the binding pattern with reference binding patterns from one or more reference binding moiety compositions to determine if the binding pattern is similar enough to meet a quality control criterion.

### Embodiment 16

A computer-implemented method of analyzing a biomolecule binding pattern against an indexed library of a substantial representation of all theoretical peptide combinations having a certain length of 3 to 12 amino acids, or a combination thereof, and being formed with a certain collection of amino acids that numbers as follows:

| | # of amino acids |
|---|---|
| Length | in collection |
| 3 | 6 to 20 |
| 4 | 4 to 20 |
| 5 | 4 to 20 |
| 6 | 4 to 19 |
| 7 | 4 to 17 |
| 8 | 4 to 16 |
| 9 | 4 to 15 |
| 10 | 4 to 14 |
| 11 | 4 to 13 |
| 12 | 4 to 12 |

wherein the maximum peptide length in the library is L; the method comprising, (a) providing to a programmed computer the binding data for the antibody composition with respect to the library peptides; (b) forming on the programmed computer an L+1 dimensional fitness space; (c) identifying on the programmed computer fitness space features comprising one or more of
the Hamming distance=1 local maximums in the data,
ridges, or
valleys;
   (d) comparing on the programmed computer the identified fitness space features with one or more second fitness space features determined from other binding data sets; (e) determining on the programmed computer (a) if the identified fitness space features correspond to a set of second fitness space features or (b) if the identified fitness space features correspond to a combination of set of second fitness space features; and (f) outputting from the computer whether (a) or (b) applies.

### Embodiment 17

A computer and laboratory-implemented method of identifying a known-to-exist binding site structure in a chemical composition known to have the binding site, the method comprising: (A) generating on an appropriately programmed computer a set of candidate binding site structures having potential to match the known-to-exist binding site structure; (B) generating a computer-generated determination of binding by, for separate such computer-generated binding site structures, on a an appropriately programmed computer; docking and determining the relative affinity of a substantial representation of all theoretical peptide combinations having a certain length; or a combination of certain lengths; and being formed with a certain collection of amino acids; (C) making a laboratory determination of binding by
contacting the chemical composition with an indexed library of a substantial representation of all such theoretical peptide combinations, and
measuring the binding of the chemical composition to substantially all of the peptides of the indexed library to derive a binding pattern; and
   (D) determining which computer-determined binding patterns sufficiently match the laboratory-determined binding pattern, thereby identifying one or more computer-generated binding sites having high probability of matching the known-to-exist binding site structure.

### Embodiment 18

The method of Embodiment 17, wherein the length of peptides used for the determination of the relative affinity of the peptide to the binding site is 3 to 5 amino acids, or a combination thereof, and being formed with a certain collection of amino acids that numbers as follows:

| | # of amino acids |
|---|---|
| Length | in collection |
| 3 | 6 or more |
| 4 | 4 or more |
| 5 | 4 or more |

### Embodiment 18

The computer and laboratory-implemented method of Embodiments 17 or 18, wherein the known-to-exist binding site structure is an antigen-binding structure of an antibody or antibody analog with known peptide sequence corresponding to an antigen-binding site.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. An indexed library on one or more solid phase supports of a substantial representation of all theoretical peptide combinations having a certain length of 3 to 5 amino acids, or a combination thereof, and being formed with a certain collection of amino acids that numbers as follows:

| | # of amino acids |
|---|---|
| Length | in collection |
| 3 | 6 to 18 |
| 4 | 4 to 18 |
| 5 | 4 to 18 |

the peptides spaced apart from the supports sufficiently such that one or more of the peptides binds an antibody composition substantially more strongly than others.

2. The indexed library of claim 1, wherein peptides of the library are made with substantially natural amino acids.

3. The indexed library of claim 1, wherein peptides of the library are on separate solid phase particles.

4. The indexed library of claim 3, wherein peptides of the library are indexed by having their respective solid phase particles be part of or connected to light-responsive transponders.

5. The indexed library of claim 1, wherein peptides of the library are made with 8 to 10 amino acids, including Arg, His, Ile, Lys, Phe, Trp, Tyr and Va.

6. A method of characterizing a first antibody composition comprising contacting the antibody composition with the indexed library of claim 1, measuring the binding of the antibody composition to a substantial representation of the theoretical peptides combinations of the indexed library, and calculating a measure of similarity to such measurements with one or more comparative antibody compositions

7. The method of characterizing of claim 6, wherein the measurement comprises identifying Hamming distance=1 local maximums.

8. The method of characterizing of claim 6, wherein the method distinguishes different monoclonal antibodies that bind the same or an overlapping site on an antigen.

9. A method of identifying a high probability for the existence of a disease with an immune response in an animal or testing the quality of a test binding biomolecule composition comprising:
contacting (i) antibodies collected from a bodily fluid of the animal or (ii) the test binding molecule composition with an indexed library of claim 1,
measuring the binding of the antibody or test binding molecule composition to substantially all of the peptides of the indexed library to derive a binding pattern, and
(i) comparing the binding pattern with reference binding patterns from animals known to have the disease and determining if there is sufficient similarity to one or more reference binding patterns to identify said high probability or (ii) comparing the binding pattern with reference binding patterns from one or more reference binding moiety compositions to determine if the binding pattern is similar enough to meet a quality control criterion.

10. The method of claim 9, wherein identifying a high probability for the existence of a disease, and wherein the disease is a cancer.

11. The method of claim 9, wherein identifying a high probability for the existence of a disease, and wherein the disease is an autoimmune disease.

12. The method of claim 9, wherein identifying a high probability for the existence of a disease, and wherein the disease is a microbial infection. (For the purposes of this application, a microbial infection is a viral, bacterial or fungal infection.)

13. The method of claim 9, wherein identifying a high probability for the existence of a disease, and wherein the disease is an allergy.

14. The method of claim 9, wherein the method is for testing the quality of a test biomolecule binding composition.

15. A computer-implemented method of analyzing a biomolecule binding pattern against an indexed library of a substantial representation of all theoretical peptide combinations having a certain length of 3 to 12 amino acids, or a combination thereof, and being formed with a certain collection of amino acids that numbers as follows:

| | # of amino acids |
|---|---|
| Length | in collection |
| 3 | 6 to 20 |
| 4 | 4 to 20 |
| 5 | 4 to 20 |
| 6 | 4 to 19 |
| 7 | 4 to 17 |
| 8 | 4 to 16 |
| 9 | 4 to 15 |
| 10 | 4 to 14 |
| 11 | 4 to 13 |
| 12 | 4 to 12 |

wherein the maximum peptide length in the library is L;
the method comprising,
providing to a programmed computer the binding data for the antibody composition with respect to the library peptides;
forming on the programmed computer an L+1 dimensional fitness space;
identifying on the programmed computer fitness space features comprising one or more of
the Hamming distance=1 local aximums in the data,
ridges; or
valleys; and
comparing on the programmed computer the identified fitness space features with one or more second fitness space features determined from other binding data sets;
determining on the programmed computer (a) if the identified fitness space features correspond to a set of second fitness space features or (b) if the identified fitness space features correspond to a combination of set of second fitness space features;
outputting from the computer whether (a) or (b) applies.

16. A computer and laboratory-implemented method of identifying a known-to-exist binding site structure in a chemical composition known to have the binding site, the method comprising:
generating on an appropriately programmed computer a set of candidate binding site structures having potential to match the known-to-exist binding site structure;
generating a computer-generated determination of binding by, for separate such computer-generated binding site structures, on a an appropriately programmed computer, docking and determining the relative affinity of a substantial representation of all theoretical peptide combinations having a certain length, or a combination of certain lengths, and being formed with a certain collection of amino acids;
making a laboratory determination of binding by
contacting the chemical composition with an indexed library of a substantial representation of all such theoretical peptide combinations, and
measuring the binding of the chemical composition to substantially all of the peptides of the indexed library to derive a binding pattern; and
determining which computer-determined binding patterns sufficiently match the laboratory-determined binding pattern, thereby identifying one or more computer-generated binding sites having high probability of matching the known-to-exist binding site structure.

17. The method of claim 16, wherein the length of peptides used for the determination of the relative affinity of the peptide to the binding site is 3 to 5 amino acids, or a combination thereof, and being formed with a certain collection of amino acids that numbers as follows:

| | # of amino acids |
|---|---|
| Length | in collection |
| 3 | 6 or more |
| 4 | 4 or more |
| 5 | 4 or more |

18. The computer and laboratory-implemented method of claim 16, wherein the known-to-exist binding site structure is an antigen-binding structure of an antibody or antibody analog with known peptide sequence corresponding to an antigen-binding site.
